(19) United States
(12) Patent Application Publication
(10) Pub. No.: US 2026/0066097 A1
(43) Pub. Date: Mar. 5, 2026

(54) ADAPTIVE SCOUT SCAN RANGE AND COMBINATION

(71) Applicant: GE Precision Healthcare LLC, Waukesha, WI (US)

(72) Inventors: Chelsey Amanda Lewis, Waukesha, WI (US); Priti Madhav, Brookfield, WI (US)

(73) Assignee: GE Precision Healthcare LLC, Waukesha, WI (US)

(21) Appl. No.: 18/820,672

(22) Filed: Aug. 30, 2024

(51) Int. Cl. G16H 30/40; A61B 6/00; A61B 6/03

(52) U.S. Cl. CPC G16H 30/40; A61B 6/032; A61B 6/54

(57) ABSTRACT

A system for combining multiple scans in a computed tomography (CT) imaging workflow, comprising a protocol selection module configured to select two or more scan protocols for a patient, a scan combination module configured to combine the scans associated with the selected scan protocols into a combined scan, wherein the scan is either contiguous or non-contiguous based on anatomical landmarks, and a scan execution module configured to control a CT scan machine to execute the combined scan on the patient.

### FIELD

A system and method for adaptive scout scan range and combination.

### BACKGROUND

Computed Tomography (CT) imaging is a diagnostic tool in the medical field. It involves the use of X-ray equipment to create detailed images of sections inside the body. A CT scan can be performed on any part of the body and is often used to diagnose diseases, monitor treatment, and guide procedures. In a typical CT imaging workflow, a technologist selects a protocol for a patient, which determines the parameters for the scan. A scout scan, also known as a topogram or localizer, is then performed to provide an overview of the area to be scanned in detail. The scout scan helps in setting the start and end locations for the diagnostic scan, which is the main scan that provides the detailed images for diagnosis.

However, in complex scanning situations or trauma scenarios, the technologist may have to select multiple protocols for a patient, each requiring a separate scout scan. This can be time-consuming and may lead to unnecessary delay in treatment and unnecessary radiation exposure if the scout scans overlap. Furthermore, the technologist may have to manually adjust the scan range to cover multiple anatomical regions of interest, which can be prone to user error and inconsistency. Additionally, the scan range may include areas of anatomy that will not be scanned for diagnosis, leading to incidental radiation exposure. These challenges can be particularly pronounced in urgent care settings where time efficiency and dose optimization are a concern.

### SUMMARY

In embodiments, the present disclosure relates to a system for combining multiple scans in a computed tomography (CT) imaging workflow, comprising a protocol selection module configured to select two or more scan protocols for a patient, a scan combination module configured to combine the scans associated with the selected scan protocols into a combined scan, wherein the scan is either contiguous or non-contiguous based on anatomical landmarks, and a scan execution module configured to control a CT scan machine to execute the combined scan on the patient.

In embodiments, the protocol selection module is further configured to automatically select the two or more scan protocols based on trauma data of the patient.

In embodiments, the trauma data comprises injuries necessitating CT scans of multiple regions of an anatomy of the patient.

In embodiments, the protocol selection module is configured to receive the two or more scan protocols from a medical professional.

In embodiments, the scan combination module is further configured to combine the scans into a contiguous scan range when the anatomical landmarks match across the selected scan protocols.

In embodiments, the scan combination module is further configured to set a start point and an end point for the contiguous scan range according to a superior value and an inferior value associated with the scan protocols.

In embodiments, the scan combination module is further configured to combine the scans into a non-contiguous scan range when the anatomical landmarks do not match across the selected scan protocols.

In embodiments, the scan combination module is further configured to set N start points and N end points for the non-contiguous scan range according to separate anatomical landmarks.

In embodiments, the scan execution module is further configured to maintain a scan dosage and window parameters for the combined scan.

In embodiments, the scan execution module is further configured to determine the anatomical landmarks from images of the patient within the CT scan machine.

In embodiments, the present disclosure relates to a method for combining multiple scans in a computed tomography (CT) imaging workflow, comprising selecting, by a protocol selection module, two or more scan protocols for a patient, combining, by a scan combination module, the scans associated with the selected scan protocols into a combined scan, wherein the scan is either contiguous or non-contiguous based on anatomical landmarks; and controlling, by a scan execution module, a CT scan machine to execute the combined scan on the patient.

In embodiments, the method comprises automatically selecting, by the protocol selection module, the two or more scan protocols based on trauma data of the patient.

In embodiments, the trauma data comprises injuries necessitating CT scans of multiple regions of an anatomy of the patient.

In embodiments, the method comprises receiving, by the protocol selection module, the two or more scan protocols from a medical professional.

In embodiments, the method comprises combining, by the scan combination module, the scans into a contiguous scan range when the anatomical landmarks match across the selected scan protocols.

In embodiments, the method comprises setting, by the scan combination module, a start point and an end point for the contiguous scan range according to a superior value and an inferior value associated with the scan protocols.

In embodiments, the method comprises combining, by the scan combination module, the scans into a non-contiguous scan range when the anatomical landmarks do not match across the selected scan protocols.

In embodiments, the method comprises setting, by the scan combination module, N start points and N end points for the non-contiguous scan range according to separate anatomical landmarks.

In embodiments, the method comprises maintaining, by the scan execution module, a scan dosage and window parameters for the combined scan.

In embodiments, the method comprises determining, by the scan execution module, the anatomical landmarks from images of the patient within the CT scan machine.

### BRIEF DESCRIPTION OF THE DRAWINGS

So that the way the above-recited features of the present disclosure may be understood in detail, a more particular description of the disclosure, briefly summarized above, may be made by reference to example embodiments, some of which are illustrated in the appended drawings. It is to be noted, however, that the appended drawings illustrate only example embodiments of this disclosure and are therefore not to be considered limiting of its scope, for the disclosure may admit to other equally effective example embodiments.

FIG. 1A illustrates a CT imaging workflow system connected via a network, according to aspects of the present disclosure.

FIG. 1B shows a block schematic diagram of an example CT imaging system, in accordance with one or more embodiments of the present disclosure.

FIG. 2 illustrates an anatomical diagram with various views of a human figure and identified anatomical landmarks, according to aspects of the present disclosure.

FIG. 3 illustrates a flowchart process for combining multiple scans in a CT imaging workflow, according to aspects of the present disclosure.

FIG. 4A depicts a table including examples of selected protocols with matching position, orientation and landmarks, and the resultant combined protocol.

FIG. 4B depicts a table including examples of selected protocols with either mismatching position and/or landmarks, and the resultant combined protocol.

FIG. 5 illustrates another flowchart process for combining multiple scans in a CT imaging workflow, according to aspects of the present disclosure.

### DETAILED DESCRIPTION

Various example embodiments of the present disclosure will now be described in detail with reference to the drawings. It should be noted that the relative arrangement of the components and steps, the numerical expressions, and the numerical values set forth in these example embodiments do not limit the scope of the present disclosure unless it is specifically stated otherwise. The following description of at least one example embodiment is merely illustrative in nature and is in no way intended to limit the disclosure, its application, or its uses.

Techniques, methods, and apparatus as known by one of ordinary skill in the relevant art may not be discussed in detail but are intended to be part of the specification where appropriate. In the examples illustrated and discussed herein, any specific values should be interpreted to be illustrative and non-limiting. Thus, other example embodiments may have different values. Notice that similar reference numerals and letters refer to similar items in the following figures, and thus once an item is defined in one figure, it is possible that it need not be further discussed for the following figures. Below, the example embodiments will be described with reference to the accompanying figures.

The present disclosure relates to a system and method for optimizing computed tomography (CT) imaging workflows, particularly in trauma scenarios or complex scanning situations where efficiency and speed are important. The disclosed technology provides an approach to managing multiple scout scans, which are initial CT images taken to determine the area to be scanned in detail.

Specifically, the system intelligently combines these multiple scout scans into either a contiguous or non-contiguous scout scan range, based on patient orientation, anatomical landmarks, and start and end locations. This workflow not only streamlines the technologist's tasks by reducing manual adjustments and prep work, but also reduces (e.g. minimizes) risk of user error and enhances consistency across different cases.

In some examples, the disclosed technology may utilize images of the patient, such as initial CT images or scout scans, to determine the areas to be scanned in detail. In some aspects, the system leverages these images to intelligently anticipate the series of scan acquisitions and combines scouts based on patient orientation, anatomical landmarks, and start and end locations. In a “camera workflow”, the system may capture and analyze images and/or video to aid in the determination of landmarks to be referenced in the scan. However, the technology also provides for a “non-camera workflow” where the user manually inputs these landmarks.

The disclosed technology offers a more efficient and user-friendly approach to protocol combination and scout scan management. By automating these processes, the disclosed technology allows for quicker acquisition times and dose savings, which may be particularly beneficial in urgent care settings where time is of the essence. The patient benefits from a more streamlined and efficient scanning process, potentially reducing their time in the scanner and their overall stress during a traumatic experience.

In a trauma scenario, where a patient requires immediate and comprehensive diagnostic imaging, the disclosed technology may be particularly beneficial. For instance, if a patient arrives with injuries that necessitate CT scans of both the head and the abdomen, the technologist may typically have to perform separate scout scans for each region. With the workflow of the disclosed technology, the system can intelligently anticipate the series of scan acquisitions that are about to be performed. If the patient's orientation, position, and anatomical landmarks for the head and abdomen match, the system can automatically combine the start and end locations for the scout scans of both regions. This results in a single, optimized scout scan range that covers both the head and the abdomen, allowing for a faster transition to the diagnostic scans.

In some cases, the patient's injuries might require non-contiguous scans, such as when the regions of interest are not adjacent to each other. The technology can handle this by creating a non-contiguous auto-scannable range that includes the specific start and end locations for each landmark, avoiding unnecessary radiation to non-targeted areas.

In some aspects, the system may utilize a predictive algorithm that analyzes a combination of patient data, historical scan data, and current scan parameters to intelligently anticipate the series of scan acquisitions. The predictive algorithm may consider factors such as the patient's medical history, the urgency of the situation, the type of trauma or condition being assessed, and the protocols typically associated with such conditions. Additionally, the system may incorporate machine learning techniques to improve its predictive capabilities over time, learning from each scan to better anticipate future scan series requirements.

The system may also employ real-time data acquisition, such as the initial scout scans or patient images, to determine the anatomical regions of interest. By analyzing these images, the system can identify anatomical landmarks and assess the patient's orientation and position, which are then used to predict the start and end locations for the scan acquisitions. This process may be further refined by the input of the technologist, who can confirm or adjust the landmarks and positions suggested by the system.

Furthermore, the system may be configured to recognize patterns in scan sequences that are commonly used in specific clinical scenarios. For example, in a trauma case involving head and abdominal injuries, the system may recognize that these regions are frequently scanned together and, therefore, anticipate that both will be included in the scan series. This pattern recognition can be based on predefined protocol combinations or derived from an analysis of past scan sequences for similar cases.

By integrating these various data sources and analytical techniques, the system can intelligently anticipate the series of scan acquisitions, thereby streamlining the scanning process and enhancing the efficiency and accuracy of the CT imaging workflow.

While the solution is described with respect to optimizing the management of scout scans, it is pertinent to note that the principles and methodologies of the disclosed technology can be extended to diagnostic scans as well. Diagnostic scans, which provide the detailed images used for medical diagnosis, can also benefit from the intelligent combination of multiple scan acquisitions.

In scenarios where a patient requires diagnostic scans of multiple regions, the system can apply the same algorithms and data analysis techniques to streamline the diagnostic scanning process. This extension to diagnostic scans further enhances the overall efficiency of the CT imaging workflow, reducing the time and radiation exposure associated with separate diagnostic scans, and potentially improving the patient experience by shortening the duration of the scanning procedure.

Moreover, the ability to intelligently combine diagnostic scans based on patient orientation, anatomical landmarks, and start and end locations can lead to a more personalized and targeted approach to patient imaging. Just as with scout scans, the system can automatically determine whether a contiguous or non-contiguous diagnostic scan range may be appropriate, thereby optimizing the scan for both diagnostic efficacy and dose efficiency. This adaptability in managing diagnostic scans underscores the versatility of the disclosed technology, making it a comprehensive solution for enhancing CT imaging workflows in a variety of clinical settings, beyond the initial scout scan phase.

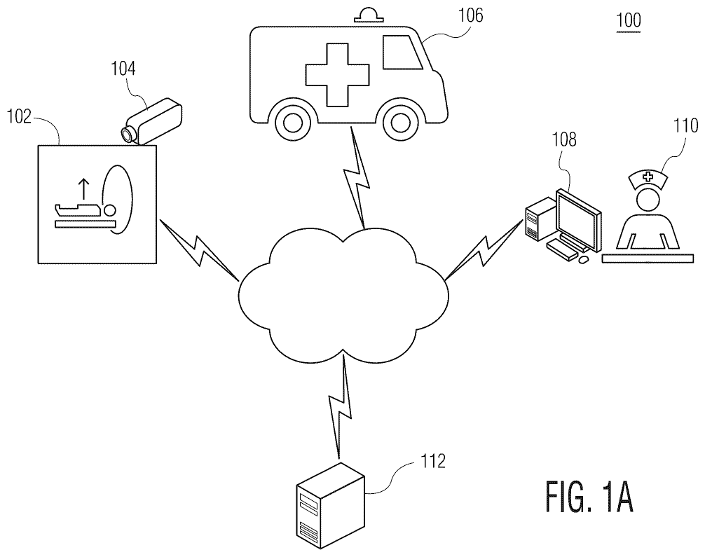

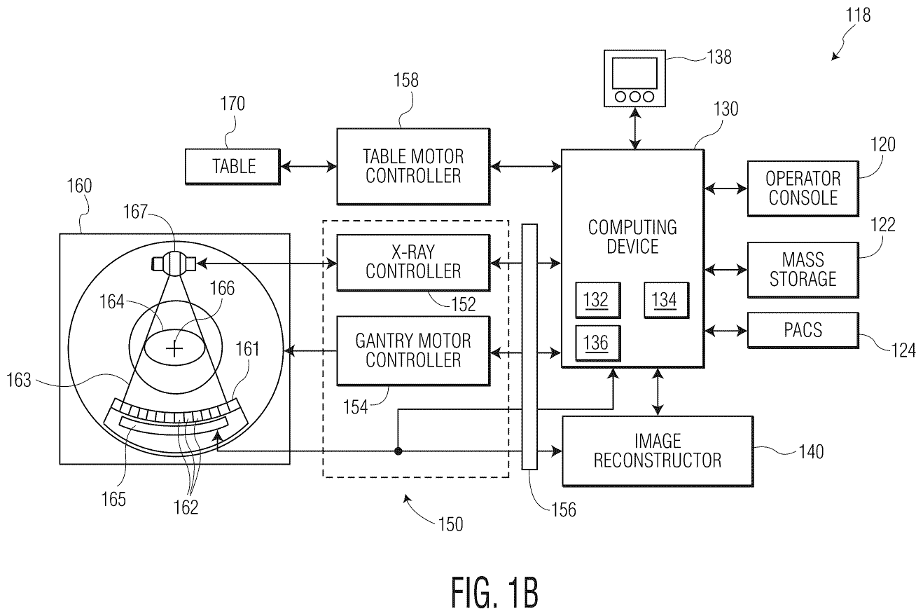

Referring now to FIG. 1A, an example CT imaging workflow system 100 is shown to include a CT scanner 102 equipped with an optional camera 104. The CT scanner 102 may be configured to perform CT scans on a patient based on selected scan protocols. The camera 104 may be used to capture images of the patient, which can be used to determine the areas to be scanned in detail. In some cases, camera 104 provides images that may be analyzed to identify regions on the patient's body that have experienced trauma, facilitating the selection of appropriate scan protocols. Additionally, the images may be analyzed to determine locations of anatomical landmarks, which are used to set the start and end points for the scout scans, ensuring precise coverage of the areas of interest. More details of the camera workflow are described with respect to later figures.

In an example, CT scanner 102, ambulance 106, workstation 108 and server 112 may be connected to one another via a cloud network 114. The ambulance 106 may be equipped with medical equipment and personnel to provide immediate medical care to the patient in a trauma situation. The cloud network 114 facilitates data exchange and communication between such that medical professional 110 in the hospital may access medical information of the patient data from ambulance 106 and from medical server 112 to help anticipate the required scout scans when the patient arrives at the hospital.

Workstation 108 allows medical professional 110 to select the scan protocols, monitor the scanning process, and review the scan results. The workstation 108 may include a user interface that allows the medical professional 110 to interact with the CT imaging workflow system 100 and control its operations. Medical server 112 may store patient data, scan protocols, scan results, and other relevant data. The server 112 may also host the software applications that control the operations of the CT imaging workflow system 100.

FIG. 1B illustrates an example imaging system 118 similar to the CT system or CT scanner 102 of FIG. 1A. In accordance with aspects of the present disclosure, the imaging system 118 is configured for imaging a subject 164. In one embodiment, the imaging system 118 may include a detector array 161. The detector array 161 may include a plurality of detector elements 162 that together sense an X-ray radiation beam 163 that passes through the subject 164 (such as a patient) to acquire corresponding projection data. In some embodiments, the detector array 161 may be fabricated in a multi-slice configuration including the plurality of rows of cells or detector elements 162, where one or more additional rows of the detector elements 162 are arranged in a parallel configuration for acquiring the projection data. The detector elements 162 may also be referred to as pixels or detector pixels.

In certain embodiments, the imaging system 118 may be configured to traverse different angular positions around the subject 164 for acquiring desired projection data. Accordingly, the gantry 160 and the components mounted thereon may be configured to rotate about a center of rotation 166 for acquiring the projection data, for example, at different energy levels. Alternatively, in embodiments where the projection angle relative to the subject 164 varies as a function of time, the mounted components may be configured to move along a general curve rather than along a segment of a circle.

As an X-ray source 167 and the detector array 161 rotate, the detector array 161 collects data of the attenuated X-ray beams. The data collected by the detector array 161 undergoes pre-processing and calibration to condition the data to represent the line integrals of the attenuation coefficients of the scanned subject 164. The processed data are commonly called projections. In some examples, the individual detectors or detector elements 162 of the detector array 161 may include photon counting detectors which register the interactions of individual photons into one or more energy bins.

The acquired sets of projection data may be used for basis material decomposition (BMD). During BMD, the measured projections are converted to a set of material-density projections. The material-density projections may be reconstructed to form a set of material-density maps or images of each respective basis material, such as bone, soft tissue, and/or contrast agent maps. The density maps or images may be, in turn, associated to form a 3D volumetric image of the basis material, for example, bone, soft tissue, and/or contrast agent, in the imaged volume.

Once reconstructed, the basis material image produced by the imaging system 118 reveals internal features of the subject 164, expressed in the densities of two basis materials. The density image may be displayed to show these features. In traditional approaches to diagnosis of medical conditions, such as disease states, and more generally of medical events, a radiologist or physician would consider a hard copy or display of the density image to discern characteristic features of interest. Such features might include lesions, sizes and shapes of particular anatomies or organs, and other features that would be discernable in the image based upon the skill and knowledge of the individual practitioner.

In one example, the imaging system 118 includes a control mechanism 150 to control movement of the components such as rotation of the gantry 160 and the operation of the X-ray source 167. In certain embodiments, the control mechanism 150 further includes an X-ray controller 152 configured to provide power and timing signals to the X-ray source 167. Additionally, the control mechanism 150 includes a gantry motor controller 154 configured to control a rotational speed and/or position of the gantry 160 based on imaging requirements.

In certain examples, the control mechanism 150 further includes a data acquisition system (DAS) 165 configured to sample analog data received from the detector elements 162 and convert the analog data to digital signals for subsequent processing. The DAS 165 may be further configured to selectively aggregate data from a subset of the detector elements 162 into so-called macro-detectors. The data sampled and digitized by the DAS 165 is transmitted to a computer or computing device 130 via a slip ring 156. In one example, the computing device 130 stores the data in a storage device or mass storage 122. The storage device 122, for example, may be any type of non-transitory memory and may include a hard disk drive, a floppy disk drive, a compact disk-read/write (CD-R/W) drive, a Digital Versatile Disc (DVD) drive, a flash drive, and/or a solid-state storage drive.

Additionally, the computing device 130 provides commands and parameters to one or more of the DAS 165, the X-ray controller 152, and the gantry motor controller 154 for controlling system operations such as data acquisition and/or processing. In certain embodiments, the computing device 130 controls system operations based on operator input. The computing device 130 receives the operator input, for example, including commands and/or scanning parameters via an operator console 120 (e.g., the workstation 108) operatively coupled to the computing device 130. The operator console 120 or workstation 108 may include a keyboard (not shown) or a touchscreen to allow the operator to specify the commands and/or scanning parameters.

Although FIG. 1B illustrates one operator console 120 or workstation 108, more than one operator console may be coupled to the imaging system 118, for example, for inputting or outputting system parameters, requesting examinations, plotting data, and/or viewing images. Further, in certain embodiments, the imaging system 118 may be coupled to multiple displays, printers, workstations, and/or similar devices located either locally or remotely, for example, within an institution or hospital, or in an entirely different location via one or more configurable wired and/or wireless networks such as the Internet and/or virtual private networks, wireless telephone networks, wireless local area networks, wired local area networks, wireless wide area networks, wired wide area networks, etc.

In one embodiment, for example, the imaging system 118 may either include, or be coupled to, a picture archiving and communications system (PACS) 124. In an exemplary implementation, the PACS 124 is further coupled to a remote system such as a radiology department information system, hospital information system, and/or to an internal or external network (not shown) to allow operators at different locations to supply commands and parameters and/or gain access to the image data.

The computing device 130 may use the operator-supplied and/or system-defined commands and parameters to operate a table motor controller 158, which in turn, may control a table 170 which may be a motorized table. Specifically, the table motor controller 158 may move the table 170 for appropriately positioning the subject 164 in the gantry 160 for acquiring projection data corresponding to the target volume of the subject 164.

As previously noted, the DAS 165 may sample and digitize the projection data acquired by the detector elements 162. Subsequently, an image reconstructor 140 may use the sampled and digitized X-ray data to perform high-speed reconstruction. Although FIG. 1B illustrates the image reconstructor 140 as a separate entity, in certain embodiments, the image reconstructor 140 may form part of the computing device 130. Alternatively, the image reconstructor 140 may be absent from the imaging system 118 and instead the computing device 130 may perform one or more functions of the image reconstructor 140. Moreover, the image reconstructor 140 may be located locally or remotely and may be operatively connected to the imaging system 118 using a wired or wireless network. Particularly, one exemplary embodiment may use computing resources in a “cloud” network cluster for the image reconstructor 140.

In one embodiment, the image reconstructor 140 stores the images reconstructed in the storage device 122. Alternatively, the image reconstructor 140 may transmit the reconstructed images to the computing device 130 to generate useful patient information for diagnosis and evaluation. In certain embodiments, the computing device 130 may transmit the reconstructed images and/or the patient information to a display or display device 138 communicatively coupled to the computing device 130 and/or the image reconstructor 140. In some embodiments, the reconstructed images may be transmitted from the computing device 130 or the image reconstructor 140 to the storage device 122 for short-term or long-term storage.

Information may be transmitted between the components residing in the gantry 160 and external devices (such as the computing device 130 and/or image reconstructor 140) via the slip ring 156, which facilitates electronic communication across the rotating gantry. In some examples, the gantry and internal components (e.g., the control mechanism 150, X-ray source 167, the detector array 161) may be collectively defined as a PCCT scanner, and as such the computing device 130 and image reconstructor 140 may reside off the scanner.

It is noted that computing device 130 executes various modules (e.g., software executing on workstation and/or server 112). These modules may include but are not limited to a protocol selection module 132, scan combination module 134 and scan execution module 136. The details of these modules are described below.

As mentioned above, the CT imaging workflow generally includes a protocol selection module 132 (e.g., software executing on workstation and/or server 112) that may be configured to select two or more scan protocols for a patient with or without manually intervention. The scan protocols can have predefined parameters set for the CT scans, such as the scan range, scan duration, and radiation dose. The protocol selection module 132 may select the scan protocols based on various factors, such as the manual input from medical professional 110, patient's medical history, the type of trauma or condition being assessed, and the protocols typically associated with such conditions.

A medical professional may select multiple protocols to address the diverse diagnostic requirements of a patient's condition, as different regions of the body may necessitate distinct imaging parameters to achieve the desired diagnostic clarity. For instance, a patient with multiple injuries may require scans with varying levels of detail, contrast, and radiation dose for each affected area. By selecting multiple protocols, the medical professional ensures that each anatomical region may be scanned according to its specific diagnostic requirements, which may include different slice thicknesses, contrast media, or scanning techniques. This tailored approach to protocol selection may be particularly valuable in emergency situations where a comprehensive and accurate diagnosis is beneficial for effective treatment planning.

In the context of the CT imaging workflow, each selected protocol may encompass multiple scan acquisitions, where each scan acquisition may be defined by specific imaging parameters tailored to distinct anatomical regions or diagnostic objectives. These scan acquisitions within a protocol can be thought of as subsets, each with its own set of instructions dictating aspects such as slice thickness, contrast levels, and radiation dose, to ensure that the resulting scans meet the precise diagnostic requirements for each area of interest. The system's ability to manage and combine these scan acquisitions intelligently across multiple protocols allows for a customized and efficient scanning process, accommodating the complex and varied imaging demands that arise in clinical practice, particularly in urgent care and trauma situations.

The CT imaging workflow may also include a scan combination module 134 (e.g., software executing on workstation and/or server 112) that may be configured to combine the scans associated with the selected scan protocols into a combined scan. The combined scan may be either contiguous or non-contiguous based on anatomical landmarks. In a contiguous scan, the scan ranges of the individual scans may be combined into a single continuous scan range. In a non-contiguous scan, the scan ranges of the individual scans may be combined into multiple separate scan ranges.

In the disclosed technology, the combination of multiple scan protocols may be achieved by integrating the parameters within the scan acquisitions defined by each protocol. Each scan acquisition represents a set of imaging parameters tailored to specific anatomical regions or diagnostic objectives, such as slice thickness, contrast levels, and radiation dose. The scan combination module 134 intelligently manages these scan acquisitions, merging them across the selected protocols to form a single combined scan that may be contiguous or non-contiguous, depending on the alignment of anatomical landmarks.

The CT imaging workflow may further include a scan execution module 136 (e.g., software executing on workstation and/or server 112) that may be configured to control the CT scanner 102 to execute the combined scan on the patient. The scan execution module 136 may control various aspects of the scanning process, such as the scan speed, scan direction, and radiation dose. The scan execution module 136 may also control the camera 104 to capture images of the patient during the scanning process.

In some cases, the protocol selection module 132 may automatically select the scan protocols based on trauma data of the patient. The trauma data may include information about the patient's injuries, such as the type and location of the injuries, the severity of the injuries, and the patient's response to the injuries. The trauma data may be obtained from various sources, such as medical records, medical imaging data, and medical sensors. The trauma data may be used to identify the regions of the patient's anatomy that require CT scanning and to select the appropriate scan protocols for these regions.

In some cases, the protocol selection module 132 may receive scan protocols that are manually selected by the medical professional 110 from the workstation 108. Once these protocols are inputted, the module intelligently combines them, taking into consideration the patient's orientation, anatomical landmarks, the desired start and end locations for the scans, and possibly other information. This intelligent combination of protocols ensures that the scans are executed in a manner that is both time-efficient and dose-optimized, further enhancing the overall effectiveness of the CT imaging workflow.

The scan execution module 136 may maintain a scan dosage and window parameters for the combined scan. This ensures that the combined scan adheres to the dosage and window parameters specified in the scan protocols, thereby maintaining the image quality and reducing (e.g. minimizing) the radiation exposure to the patient.

In some cases, the scan execution module 136 may determine the anatomical landmarks from images of the patient within the CT scan machine. The images may be captured by the camera 104 or obtained from other sources, such as medical imaging data or medical sensors. The anatomical landmarks may include various points or regions on the patient's body that are used as reference points for the CT scans. By determining the anatomical landmarks, the scan execution module 136 can accurately set the start and end locations for the combined scan, ensuring that the scan covers the intended regions of the patient's anatomy.

In a variation of the disclosed technology, the protocol selection module 132 may receive the two or more scan protocols from a medical professional 110. The medical professional 110 may select the scan protocols based on various factors, such as the patient's medical history, the type of trauma or condition being assessed, and the protocols typically associated with such conditions. This allows the medical professional 110 to customize the CT scans to the specific requirements of the patient, thereby enhancing the diagnostic accuracy and efficiency of the CT imaging workflow system 100.

As mentioned above, the imaging workflow may utilize a predictive algorithm that analyzes a combination of patient data, historical scan data, and current scan parameters to intelligently anticipate the series of scan acquisitions. The predictive algorithm may consider factors such as the patient's medical history, the urgency of the situation, the type of trauma or condition being assessed, and the protocols typically associated with such conditions. This allows the CT imaging workflow to customize the CT scans to the specific requirements of the patient, thereby enhancing the diagnostic accuracy and efficiency of the system.

In some cases, the CT imaging workflow system 100 may incorporate machine learning techniques to improve its predictive capabilities over time. The machine learning techniques may involve training a machine learning model using historical scan data and patient data. The trained model may then be used to predict the series of scan acquisitions for a new patient based on their medical history and current scan parameters. This learning from each scan allows the CT imaging workflow to better anticipate future scan series requirements, thereby further enhancing the efficiency and accuracy of the system.

In some aspects, the machine learning models that can be used include convolutional neural networks (CNNs) for image recognition tasks, which can identify anatomical landmarks and assess injuries from CT images. Additionally, recurrent neural networks (RNNs) or long short-term memory (LSTM) networks may be employed to analyze sequential patient data and predict the series of scan acquisitions based on historical patterns and current patient conditions.

Furthermore, the CT imaging workflow may be configured to recognize patterns in scan sequences that are commonly used in specific clinical scenarios. For example, in a trauma case involving head and abdominal injuries, the system may recognize that these regions are frequently scanned together and, therefore, anticipate that both will be included in the scan series. This pattern recognition can be based on predefined protocol combinations or derived from an analysis of past scan sequences for similar cases. By recognizing these patterns, the CT imaging workflow can streamline the scanning process and reduce the time and effort spent on manual adjustments and prep work.

In some aspects, the predictive algorithm, machine learning techniques, and pattern recognition capabilities of the CT imaging workflow may be implemented in the protocol selection module 132, the scan combination module 134, or the scan execution module 136. These modules may work together to select the scan protocols, combine the scans, and execute the combined scan, thereby providing a comprehensive solution for optimizing CT imaging workflows. The modules described herein such as the protocol selection module 132, scan combination module 134, and scan execution module 136 may be software modules that execute on one or more processors of the system devices, such as the workstation and server.

Consider a use case example where upon admission to the hospital, a patient with suspected injuries to both the head and abdomen presents a complex scanning scenario. The medical professional 110, utilizing the workstation 108, selects two or more distinct scout scans tailored to each region of interest. The scan combination module 134, leveraging its intelligent design, recognizes that the anatomical landmarks for both the head and abdomen are aligned and thus, combines the two separate scout scans into a single contiguous scan range.

For example, as the patient is positioned within the CT scanner 102, the camera 104 captures real-time images of the patient, which are then analyzed by the scan execution module 136. The system may identify the precise anatomical landmarks based on the camera images, such as the Orbital Meatal Line for the head and the Xyphoid Process for the abdomen. With the landmarks determined, the scan execution module 136 adjusts the scanner parameters accordingly and proceeds to execute the optimized contiguous scan.

In the described use case, once the anatomical landmarks are identified and the contiguous scan range may be established, the scan execution module 136 selects the scan parameters to ensure image quality and patient safety. The system may choose the greatest kilovoltage (kV) and milliampere (mA) settings from among the selected protocols. This approach ensures that the scan has sufficient energy to penetrate the targeted anatomical regions, providing clear and detailed images for diagnosis. Additionally, the system may standardize the window width and window level across the combined scan range. The window width and level are parameters that affect the display of the CT images, with the window width determining the range of CT numbers to display and the window level setting the midpoint of this range. By maintaining a common window width and level, the system ensures consistent image contrast and brightness, which may be particularly beneficial when interpreting scans from multiple regions in a single contiguous range. More details of parameter selection and combination are described below.

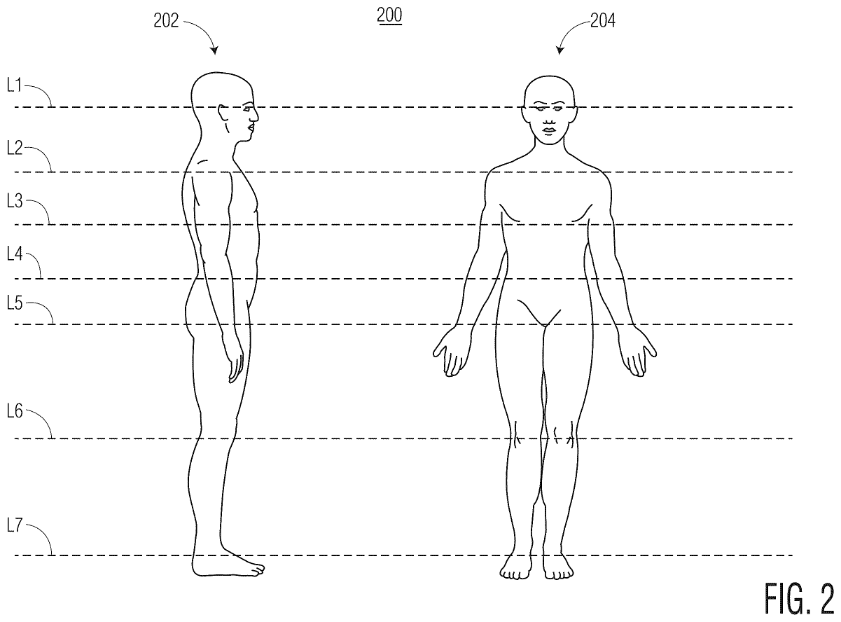

In order to provide some anatomical context to the CT scan parameters, consider anatomical diagram 200 in FIG. 2 which shows several anatomical landmarks for human positions 202 and 204. These anatomical landmarks include but not limited to the Orbital Meatal Line L1, Sternal Notch L2, Xyphoid Process L3, Illiac Crest L4, Symphysis Pubis L5, Knee L6, and Ankle L7. These landmarks serve as reference points for the CT scans, helping to define the scan range and guide the positioning of the patient. In some cases, the trauma data of the patient may include injuries necessitating CT scans of multiple regions of the patient's anatomy. These regions may correspond to the anatomical landmarks identified in the anatomical diagram 200.

In some aspects, the scan combination module 134 of the CT imaging workflow may combine the scans associated with the selected scan protocols into a contiguous scan range when the anatomical landmarks match across the selected scan protocols. For instance, if the patient's orientation, position, and anatomical landmarks for the head and abdomen match, the system can automatically combine the start and end locations for the scout scans of both regions. This results in a single, optimized scout scan range that covers both the head and the abdomen, allowing for a faster transition to the diagnostic scans.

The scan combination module 134 may further set a start point and an end point for the contiguous scan range according to a superior value and an inferior value associated with the scan protocols. This ensures that the combined scan covers the intended regions of the patient's anatomy, from the superior-most point to the inferior-most point, thereby maximizing the diagnostic value of the scan.

In some cases, the anatomical landmarks may not match across the selected scan protocols, necessitating non-contiguous scans. For example, the regions of interest may not be adjacent to each other, such as when the head and the abdomen are to be scanned separately. In such cases, the scan combination module 134 may combine the scans into a non-contiguous scan range. This involves setting a number of N start points and a number of N end points for the non-contiguous scan range according to separate anatomical landmarks. This approach allows the system to perform separate scans for each region of interest, avoiding unnecessary radiation to non-targeted areas and optimizing dose efficiency.

These variations in the scan combination process, whether resulting in a contiguous or non-contiguous scan range, demonstrate the flexibility and adaptability of the CT imaging workflow in handling different scanning scenarios. By intelligently combining multiple scout scans based on patient orientation, anatomical landmarks, and start and end locations, the system can streamline the scanning process, enhance efficiency, and optimize dose usage, thereby providing a more effective and user-friendly solution for CT imaging workflows.

The subsequent flowcharts provide a detailed operational view of the CT imaging workflow, illustrating the step-by-step process for combining multiple scout scans into a single, optimized scan range. These flowcharts depict both manual and automated pathways for protocol prediction, landmark identification, and scan execution, showcasing the system's adaptability to various clinical scenarios. Through these visual representations, the intricate mechanisms by which the system intelligently anticipates and executes scan acquisitions are elucidated, further highlighting the approach to enhancing diagnostic accuracy and efficiency in urgent care and complex scanning situations.

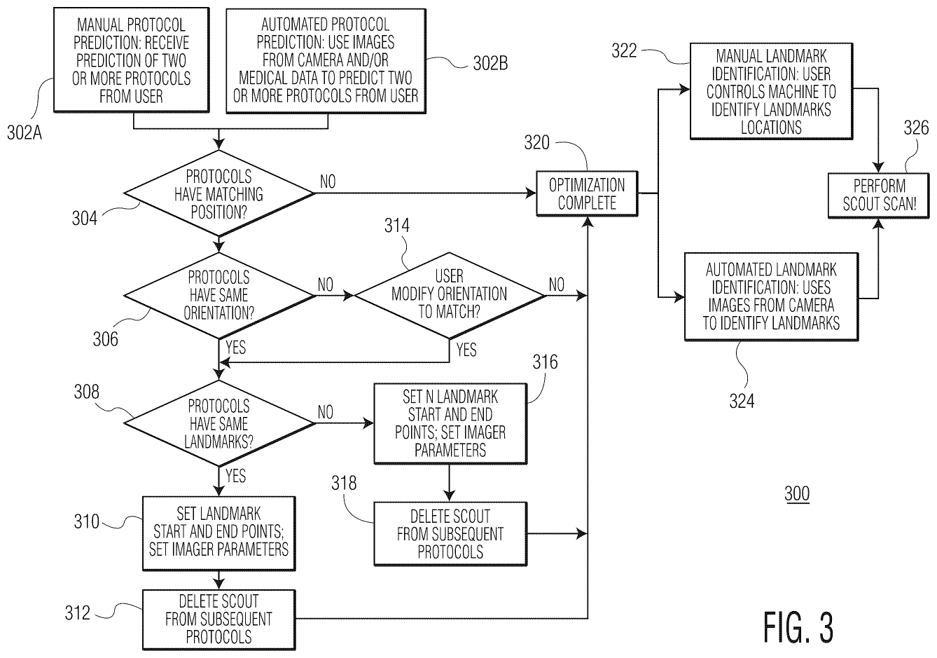

Referring to FIG. 3, the flowchart process 300 illustrates the steps involved in combining multiple scans in a CT imaging workflow. The process begins with either a manual protocol prediction step 302A, where the system receives predictions of two or more protocols manually entered by the medical professional, or an automated protocol prediction step 302B, which uses images from a camera and/or medical data to predict two or more protocols.

In the manual protocol prediction step 302A, the user, such as a medical professional 110, may select the scan protocols based on various factors, such as the patient's medical history, the type of trauma or condition being assessed, and the protocols typically associated with such conditions. This allows the user to customize the CT scans to the specific requirements of the patient, thereby enhancing the diagnostic accuracy and efficiency of the CT imaging workflow.

In the automated protocol prediction step 302B, the CT imaging workflow may automatically select the scan protocols based on trauma data of the patient. The trauma data may include information about the patient's injuries, such as the type and location of the injuries, the severity of the injuries, the patient's response to the injuries, and possibly images of the trauma. The trauma data may be used to identify the regions of the patient's anatomy that require CT scanning and to select the appropriate scan protocols for these regions.

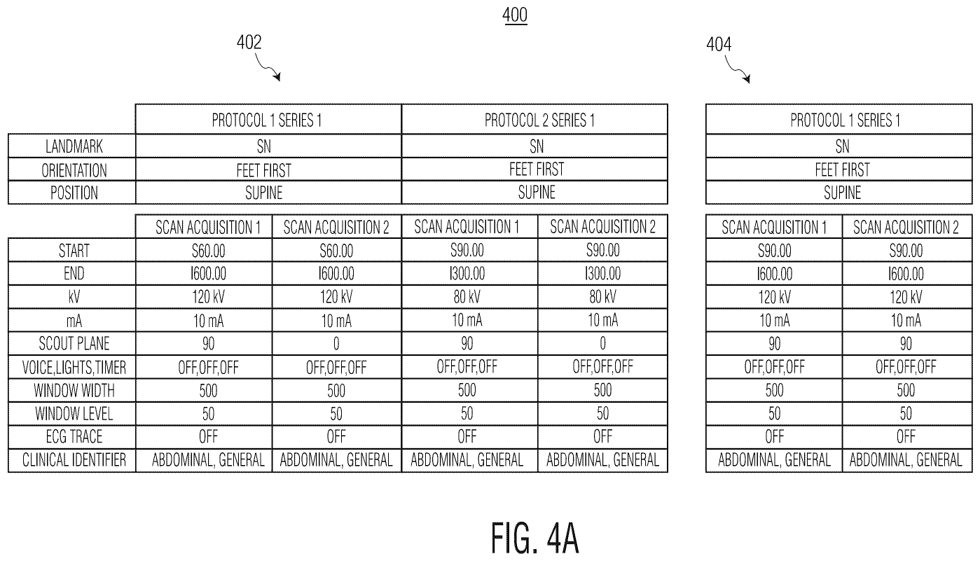

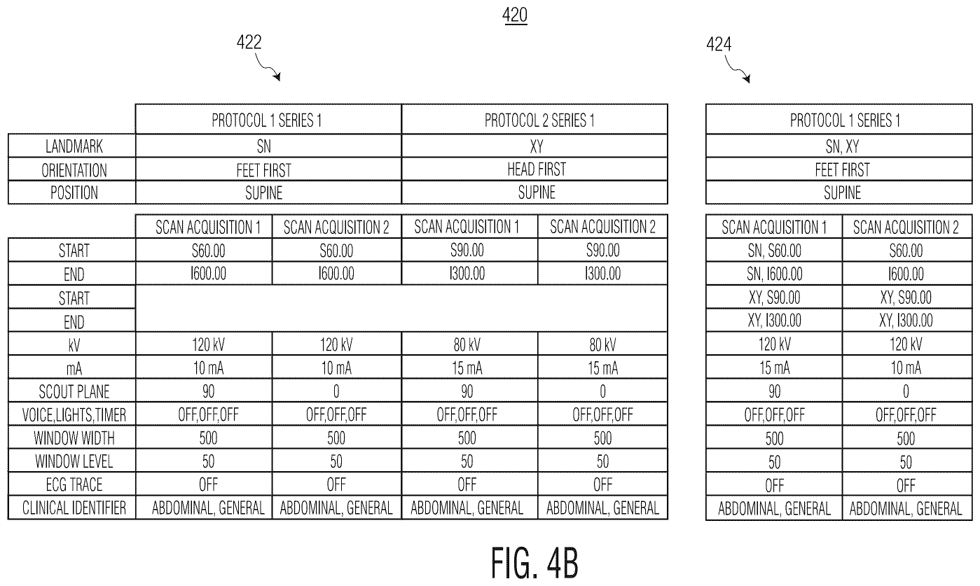

Following the protocol prediction, the process checks if the protocols have matching positions in step 304. If they do match, it proceeds to check if the protocols have the same orientation in step 306. If the orientations match, it further checks if the protocols have the same landmarks in step 308. If the conditions are met, the system sets the landmark start and end points (contiguous scan with same points) and sets the scanner parameters in step 310, then deletes the scout from subsequent protocols in step 312, leading to optimization completion step 320. An example of selected protocols with matching position, orientation and landmarks, and the resultant combined protocol are shown in table 400 of FIG. 4A where section 402 shows two selected protocols each with two scan acquisitions, and section 404 shows the optimized protocol with two scan acquisitions.

In the example shown in table 400, the optimization of CT imaging workflows, the system intelligently selects the ideal parameters from the scan acquisitions defined by the selected scan protocols. This selection process may be guided by the principle of maximizing diagnostic efficacy while ensuring patient safety.

For example, the starting point for the combined scan may be chosen based on the superior-most anatomical landmark across the protocols, ensuring comprehensive coverage of the targeted regions from the topmost point. Conversely, the end point may be determined by the inferior-most landmark, thereby encompassing the full extent of the areas of interest. This superior-to-inferior range guarantees that no relevant anatomy is omitted from the scan.

In this example, the system further refines the scan parameters by selecting the kV and mA values (e.g., greatest values) from the protocol scan acquisitions. This ensures that the scans have sufficient energy to penetrate the targeted regions, providing clear and detailed images for diagnosis. Each anatomical plane identified in the protocols may be treated as a distinct scan acquisition, allowing for tailored imaging of each specific region. Consistency across the combined scan range may be achieved by maintaining the same window width and level, which define the display characteristics of the CT images, such as contrast and brightness.

Also, if any of the protocols have lighting or timers activated, these features may be kept on to preserve the integrity of the scanning process. Similarly, if an ECG trace may be active in any of the protocol scan acquisitions, it may remain on during the combined scan to ensure continuous cardiac monitoring. Lastly, a common clinical identifier may be used throughout the combined scan to facilitate the tracking and documentation of the imaging process, enhancing the system's efficiency and the medical professional's ability to manage patient data effectively.

In addition to the described methods, the system may select the optimum parameters for the combined scan by analyzing patient-specific factors such as age, weight, and known medical conditions, which can influence the appropriate radiation dose and image resolution. The system may also consider the urgency of the clinical situation, adjusting parameters to prioritize speed over resolution in life-threatening scenarios.

Furthermore, the system may employ advanced algorithms that analyze previous successful scans for similar cases to suggest the ideal parameters, thereby leveraging historical data to inform current decisions. Additionally, the system may incorporate feedback mechanisms, allowing the medical professional to refine the parameters based on real-time visual and diagnostic feedback during the scan, ensuring that the parameters are dynamically optimized for the specific patient and clinical context.

In another example, if the protocols do not have matching positions, the process checks if the user modifies the orientation to match in step 314. If the user modifies the orientation, the process continues to check if the protocols have the same landmarks in step 308. If the landmarks do not match, the system sets N landmark start and end points and sets the scanner parameters in step 316 (two or more scans with different points), then deletes the scout from subsequent protocols in step 318, leading to optimization completion step 320.

An example of selected protocols with either mismatching position and/or landmarks, and the resultant combined protocol are shown in table 420 of FIG. 4B where section 422 shows two selected protocols each with two scan acquisitions, and section 424 shows the optimized protocol with two scan acquisitions.

In the pursuit of optimizing CT imaging workflows, the system employs a strategic approach to parameter selection from the protocol scan acquisitions designated for each patient. This involves the choice of multiple distinct anatomical landmarks from each scan acquisition, which serve as precise reference points for the scans. By identifying and utilizing these landmarks, the system ensures that each region of interest is accurately targeted, enhancing the diagnostic utility of the scans. Furthermore, the system selects the greatest kV and mA values available within the protocol scan acquisitions. This selection may be beneficial as it guarantees that the scans possess the requisite energy to penetrate the targeted anatomical regions, thereby yielding clear and detailed images that are beneficial for accurate diagnosis.

The system may also adopt an intelligent approach to managing the imaging parameters by treating each anatomical plane as a distinct scan acquisition. This allows for the customization of imaging parameters to suit the specific requirements of each anatomical region, ensuring that the scans are both precise and tailored to the patient's diagnostic needs. Consistency in image quality may be maintained by keeping the window width and level uniform across the combined scan range. This uniformity ensures that the contrast and brightness of the CT images remain consistent, which may be particularly beneficial when interpreting scans that encompass multiple regions. Additionally, the system preserves any activated lighting or timers from the protocol scan acquisitions, maintaining the integrity of the scanning process. If an ECG trace is active in any of the protocol scan acquisitions, it may be kept on during the combined scan to facilitate continuous cardiac monitoring. Lastly, the system employs a common clinical identifier throughout the combined scan, which streamlines the tracking and documentation of the imaging process, thereby enhancing the efficiency of the system and the medical professional's ability to manage patient data effectively.

In selecting parameters for a multi-landmark scan, the system may also utilize a patient's real-time physiological data, such as heart rate or respiratory cycle, to adjust scan timing for motion-sensitive regions, thereby reducing artifacts and improving image clarity. As mentioned above, machine learning can be employed to predict the ideal scan parameters based on a database of similar patient scans, optimizing for image quality and radiation dose. Additionally, the system may use contrast-enhancement prediction models to determine the appropriate timing and concentration of contrast agents for enhanced visualization of specific anatomical structures. The integration of these advanced techniques ensures that the multi-landmark scanning process may be tailored to the individual patient's condition and the specific diagnostic requirements of each case.

It is noted that the process in FIG. 3 also includes manual landmark identification step 322, where the user may be able to control the machine to identify landmark locations, and automated landmark identification step 324, which may use images from the camera to identify landmarks. Both identification methods lead to performing the scout scan step 326.

In some aspects, the manual landmark identification step 322 and the automated landmark identification step 324 represent alternative use cases for identifying anatomical landmarks within the CT imaging workflow. In the manual landmark identification step 322, the user, such as a medical professional, actively engages with the CT scan machine's interface to pinpoint and mark the locations of specific anatomical landmarks on the patient's body. This manual process allows for user discretion and expertise to be applied when determining the relevant landmarks for the scan. Conversely, the automated landmark identification step 324 leverages advanced imaging technology, where the camera integrated with the CT scanner captures images of the patient. These images are then analyzed by software algorithms designed to automatically detect and identify the anatomical landmarks without the direct intervention of the user. This automated process can increase efficiency and reduce the potential for human error, particularly in urgent care scenarios where time is of the essence. Both the manual and automated landmark identification methods culminate in the performance of the scout scan step 326, where the CT scanner acquires preliminary images, known as scout scans, based on the identified landmarks.

In a use case scenario, consider a patient admitted to the emergency department with multiple trauma injuries, requiring immediate and comprehensive diagnostic imaging. The medical professional 110, through the workstation 108, selects distinct scan protocols for the head and the lower extremities, which are not adjacent anatomical regions and therefore cannot be covered by a contiguous scan range. The scan combination module 134, upon receiving the selected protocols, identifies that the anatomical landmarks for the head and the lower extremities do not align, necessitating a non-contiguous scan approach. The scan combination module 134 may then set separate N start points and N end points for the non-contiguous scan range in step 316, ensuring that the CT scanner 102 performs individual scans for each targeted region without exposing non-relevant areas to radiation.

Once the non-contiguous scan range is determined, the scan execution module 136 adjusts the scanner parameters to optimize image quality and radiation dose for each separate scan. The system may select the appropriate kV and mA settings (and various other settings as shown in the tables) for each protocol, ensuring that the scans are performed with sufficient energy to penetrate the targeted regions while maintaining patient safety. The scan execution module 136 generates optimized non-contiguous scans in step 320 and uses a camera to capture images of the patient in step 324 to identify relevant anatomical landmarks for the head, such as the Orbital Meatal Line L1, and for the lower extremities, such as the Knee L6 and Ankle L7. Once identified, the scans are performed in step 326.

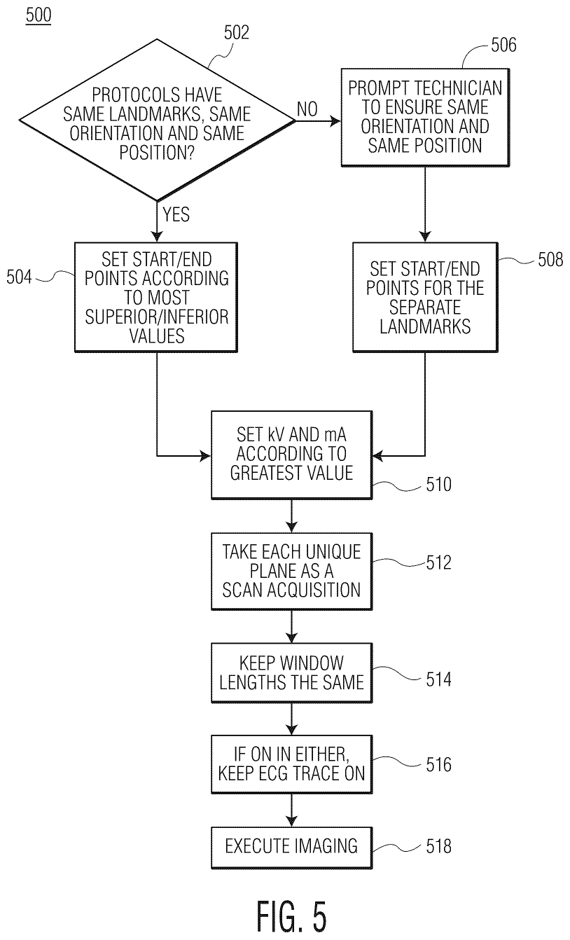

Referring to FIG. 5, the flowchart process 500 illustrates the steps involved in selecting appropriate parameters when combining multiple scans in a CT imaging workflow. The process begins with a protocol comparison step 502, which checks if the protocols have the same landmarks, orientation, and position. If they do, the process proceeds to setting start/end points according to superior/inferior values in step 504. For example, the starting position may be taken as the most superior value, whereas the end position may be taken as the most inferior position. However, if the protocols do not match, the process then prompts the technician to ensure the same orientation and position in step 506. Following this, the setting start/end points for separate landmarks step 508 is executed. For example, a first landmark may indicate a first start position from which a first end position may be set by comparison. Likewise, a second landmark may indicate a second start position from which a second end position may be set by comparison. In either case, the process then continues to a process box for setting kV and mA values in step 510. In one example, the kV and mA values may be selected as the greatest values (e.g., the greatest kV and mA are chosen from the initially selected protocols and set for the combined scan). Next, taking each unique plane as a scan acquisition in step 512 is executed, followed by keeping window length the same in step 514. The process may also check if an ECG trace is on in either protocol and keeps it on if so, as shown in step 516. The process may conclude with an executing imaging step 518.

In some variations of the disclosed technology, the system may handle situations where the protocols do not have matching positions, orientations, or landmarks. In such cases, the system may prompt the technician to ensure the same orientation and position, as indicated by the prompting technician to ensure same orientation and position step 506. If the technician confirms the same orientation and position, the system may proceed to set the start and end points for the separate landmarks, as indicated by the setting start/end points for separate landmarks step 508. This allows the system to handle non-contiguous scans, where the regions of interest are not adjacent to each other.

Consider a patient who has been involved in a high-impact vehicle accident and is suspected to have sustained injuries to both the cervical spine and the pelvis. The medical professional, using the workstation, selects two distinct scan protocols-one for the cervical spine and another for the pelvis. Due to the urgency of the situation and the non-adjacency of the anatomical regions, a non-contiguous scan may be deemed appropriate.

In this example, the protocol comparison step 502 reveals that the selected protocols do not have the same landmarks, orientation, or position. The technician may be prompted to ensure the same orientation and position for the patient in step 506, which is achieved by repositioning the patient on the CT scanner bed. Once the patient is correctly positioned, the setting start/end points for separate landmarks step 508 is executed. The cervical spine is identified using the Sternal Notch L2 as the superior landmark and the Xyphoid Process L3 as the inferior landmark, while the pelvis is identified using the Illiac Crest L4 as the superior landmark and the Symphysis Pubis L5 as the inferior landmark.

The system may then proceed to the setting kV and mA according to the greatest value in step 510, where the kV and mA values are selected as the greatest values from the initially selected protocols. This ensures that the imaging is performed with sufficient energy to penetrate the targeted regions while maintaining patient safety. Taking each unique plane as a scan acquisition in step 512 is executed to ensure that each anatomical region is treated as a separate scan acquisition for imaging purposes.

Keeping window lengths the same in step 514 standardizes the window width and level across the separate scans, ensuring consistent image quality. The system checks if an ECG trace is on in either protocol in step 516 and maintains it on for the imaging process if it is. The executing imaging step 518 is then carried out, where the CT scanner performs the non-contiguous scans of the cervical spine and pelvis, providing the medical team with the diagnostic images they require to assess the patient's injuries and plan the appropriate treatment.

In addition to setting the start and end points for the combined scan, the system may also configure other parameters such as clinical identifiers, lighting settings, timers, voice command capabilities, and any other options available in the original protocols. These parameters can be tailored to the specific requirements of each scan protocol and the patient's condition, ensuring a personalized and efficient scanning experience. Clinical identifiers may include patient name, identification number, or other relevant information that assists in tracking and documenting the scan process. Lighting settings can be adjusted to enhance the visibility of anatomical landmarks or to create a more comfortable environment for the patient. Timers may be used to synchronize the scan with other procedures or to monitor the duration of the scan for efficiency. Voice commands can provide hands-free operation, allowing the technologist to maintain sterility or manage multiple tasks simultaneously. By integrating these additional parameters into the combined scan, the system further customizes the scanning process to meet the diverse and complex demands of modern medical imaging workflows.

While the foregoing is directed to example embodiments described herein, other and further example embodiments may be devised without departing from the basic scope thereof. For example, aspects of the present disclosure may be implemented in hardware or software or a combination of hardware and software. One example embodiment described herein may be implemented as a program product for use with a computer system. The program(s) of the program product defines functions of the example embodiments (including the methods described herein) and may be contained on a variety of computer-readable storage media. Illustrative computer-readable storage media include, but are not limited to: (i) non-writable storage media (e.g., read-only memory (ROM) devices within a computer, such as CD-ROM disks readably by a CD-ROM drive, flash memory, ROM chips, or any type of solid-state non-volatile memory) on which information is permanently stored; and (ii) writable storage media (e.g., floppy disks within a diskette drive or hard-disk drive or any type of solid-state random-access memory) on which alterable information is stored. Such computer-readable storage media, when carrying computer-readable instructions that direct the functions of the disclosed example embodiments, are example embodiments of the present disclosure.

It will be appreciated by those skilled in the art that the preceding examples are exemplary and not limiting. It is intended that all permutations, enhancements, equivalents, and improvements thereto are apparent to those skilled in the art upon a reading of the specification and a study of the drawings are included within the true spirit and scope of the present disclosure. It is therefore intended that the following appended claims include all such modifications, permutations, and equivalents as fall within the true spirit and scope of these teachings.

What is claimed:

1. A system for combining multiple scans in a computed tomography (CT) imaging workflow, comprising:
a protocol selection module configured to select two or more scan protocols for a patient;
a scan combination module configured to combine the scans associated with the selected scan protocols into a combined scan, wherein the scan is either contiguous or non-contiguous based on anatomical landmarks; and
a scan execution module configured to control a CT scan machine to execute the combined scan on the patient.

2. The system of claim 1, wherein the protocol selection module is further configured to automatically select the two or more scan protocols based on trauma data of the patient.

3. The system of claim 2, wherein the trauma data comprises injuries necessitating CT scans of multiple regions of an anatomy of the patient.

4. The system of claim 1, wherein the protocol selection module is configured to receive the two or more scan protocols from a medical professional.

5. The system of claim 1, wherein the scan combination module is further configured to combine the scans into a contiguous scan range when the anatomical landmarks match across the selected scan protocols.

6. The system of claim 5, wherein the scan combination module is further configured to set a start point and an end point for the contiguous scan range according to a superior value and an inferior value associated with the scan protocols.

7. The system of claim 1, wherein the scan combination module is further configured to combine the scans into a non-contiguous scan range when the anatomical landmarks do not match across the selected scan protocols.

8. The system of claim 7, wherein the scan combination module is further configured to set N start points and N end points for the non-contiguous scan range according to separate anatomical landmarks.

9. The system of claim 1, wherein the scan execution module is further configured to maintain a scan dosage and window parameters for the combined scan.

10. The system of claim 1, wherein the scan execution module is further configured to determine the anatomical landmarks from images of the patient within the CT scan machine.

11. A method for combining multiple scans in a computed tomography (CT) imaging workflow, comprising:
selecting, by a protocol selection module, two or more scan protocols for a patient;
combining, by a scan combination module, the scans associated with the selected scan protocols into a combined scan, wherein the scan is either contiguous or non-contiguous based on anatomical landmarks; and
controlling, by a scan execution module, a CT scan machine to execute the combined scan on the patient.

12. The method of claim 11, further comprising:
automatically selecting, by the protocol selection module, the two or more scan protocols based on trauma data of the patient.

13. The method of claim 12, wherein the trauma data comprises injuries necessitating CT scans of multiple regions of an anatomy of the patient.

14. The method of claim 11, further comprising:
receiving, by the protocol selection module, the two or more scan protocols from a medical professional.

15. The method of claim 11, further comprising:
combining, by the scan combination module, the scans into a contiguous scan range when the anatomical landmarks match across the selected scan protocols.

16. The method of claim 15, further comprising:
setting, by the scan combination module, a start point and an end point for the contiguous scan range according to a superior value and an inferior value associated with the scan protocols.

17. The method of claim 11, further comprising:
combining, by the scan combination module, the scans into a non-contiguous scan range when the anatomical landmarks do not match across the selected scan protocols.

18. The method of claim 17, further comprising:
setting, by the scan combination module, N start points and N end points for the non-contiguous scan range according to separate anatomical landmarks.

19. The method of claim 11, further comprising:
maintaining, by the scan execution module, a scan dosage and window parameters for the combined scan.

20. The method of claim 11, further comprising:
determining, by the scan execution module, the anatomical landmarks from images of the patient within the CT scan machine.
